(19) United States
(12) Patent Application Publication
(10) Pub. No.: US 2024/0420800 A1
(43) Pub. Date: Dec. 19, 2024

(54) METHOD FOR HRD DETECTION IN TARGETED cfDNA SAMPLES USING DE NOVO MUTATIONAL SIGNATURES

(71) Applicant: GUARDANT HEALTH, INC., Palo Alto, CA (US)

(72) Inventors: Denis TOLKUNOV, Dublin, CA (US); Catalin BARBACIORU, Fremont, CA (US)

(21) Appl. No.: 18/743,839

(22) Filed: Jun. 14, 2024

Related U.S. Application Data
(60) Provisional application No. 63/508,462, filed on Jun. 15, 2023.

(51) Int. Cl. G16B 20/20; G16H 50/50

(52) U.S. Cl. CPC G16B 20/20; G16H 50/50

(57) ABSTRACT

Described herein are method for determining homologous recombination repair deficiency (HRD) status in a subject, including use of samples containing cell free nucleic acid such as cell free DNA (cfDNA). As such nucleic acid in samples such as blood are small quantities, described herein are techniques to analyze signatures present in cell free nucleic acids to provide metrics related to the presence or absence of a homologous recombination repair deficiency in a given subject.

### CROSS-REFERENCE TO RELATED APPLICATIONS

This application claims the benefit of priority of U.S. Provisional Patent Application No. 63/508,462, filed Jun. 15, 2023, which is incorporated by reference herein in its entirety for all purposes.

### BACKGROUND

Cancer is a major cause of disease worldwide. Each year, tens of millions of people are diagnosed with cancer around the world, and more than half eventually die from it. In many countries, cancer ranks the second most common cause of death following cardiovascular diseases. Early detection is associated with improved outcomes for many cancers.

Cancer can be caused by the accumulation of genetics variations within an individual's normal cells, at least some of which result in improperly regulated cell division. Such variations commonly include copy number variations (CNVs), single nucleotide variations (SNVs), gene fusions, insertions and/or deletions (indels), epigenetic variations including 5-methylation of cytosine (5-methylcytosine) and association of DNA with chromatin and transcription factors.

Cancers are often detected by biopsies of tumors followed by analysis of cells, markers or DNA extracted from cells. But more recently it has been proposed that cancers can also be detected from cell-free nucleic acids in body fluids, such as blood or urine. Such tests have the advantage that they are noninvasive and can be performed without identifying suspected cancer cells in biopsy. However, such tests are complicated by the fact that the amount of nucleic acids in body fluids is very low and what nucleic acids are present are heterogeneous in form (e.g., RNA and DNA, single-stranded and double-stranded, and various states of post-replication modification and association with proteins, such as histones).

While signatures exist for cancer detection, it is often the case that the original mutational signatures were obtained from whole genome sequence (WGS) data. The distribution of the mutations and therefore the shape of the signature can be substantially different for a targeted panel, as is often deployed for cfDNA based detection. Here, the number of somatic SNVs that we detect in the target regions is small and it may not be sufficient to detect the signature in a single sample.

Thus, there is a need for improved systems and methods for improved cancer detection using liquid biopsy assays. Described herein is a platform for detection of homologous recombination deficiency using de novo mutational signatures in a combination with a machine learning approach, including application in targeted cfDNA samples. The described computer- implemented systems and methods have improved capability to classify a sample as containing tumor-derived DNA with heightened sensitivity.

### BRIEF DESCRIPTION OF THE DRAWINGS

The accompanying drawings, which are incorporated in and constitute a part of this specification, illustrate certain implementations, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1. SNV signature SBS3 (Signature 3) Single base substitutions (SBS) signatures identified using 96 different contexts, considering the mutated base (six substitution subtypes: C>A, C>G, C>T, T>A, T>C, and T>G) and the bases immediately 5′ and 3′. SBS3 has been proposed as a predictor of defective homologous recombination-based repair. Signature 3 is strongly associated with germline and somatic biallelic inactivation of BRCA1 and BRCA2. In pancreatic cancer, responders to platinum therapy usually exhibit SBS3 mutations. In breast cancer Signature 3 outperforms LOH and LST scores in identifying samples with events in HR-pathway genes: BRCA1, BRCA2, RAD51C, and PALB2 (AUC: 0.8-0.9).*

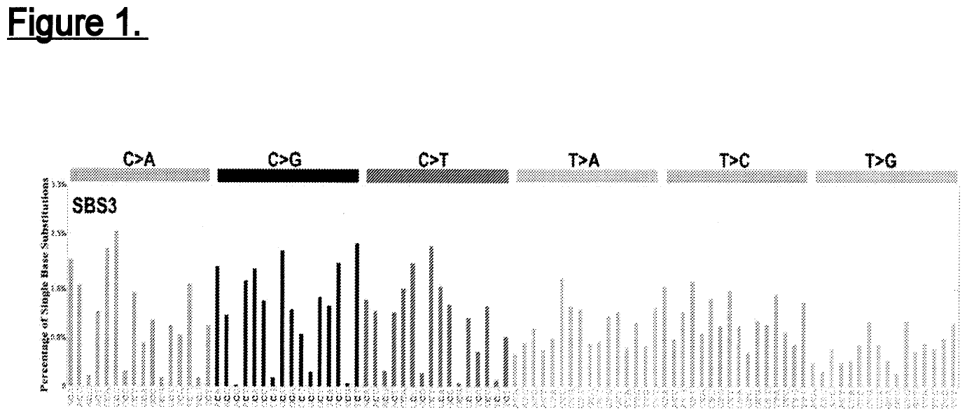

FIG. 2. Extraction of de novo mutational signatures. Select samples with more than 10 somatic SNVs. Build mutational count matrix (96 SBS types×samples). Extract de novo mutational signatures for HRD+ and HRD− samples. Search for an optimal solution between 1 and 10 mutational signatures. For each rank perform 100 independent NMFs of the normalized Poisson resampled input matrices. Identify the optimal factorization rank by simultaneously maximizing the stability and minimizing the reconstruction error. To reveal the actiology of the de novo signatures they are decomposed to known COSMIC signatures. The decomposition is based on the non-negative least squares algorithm

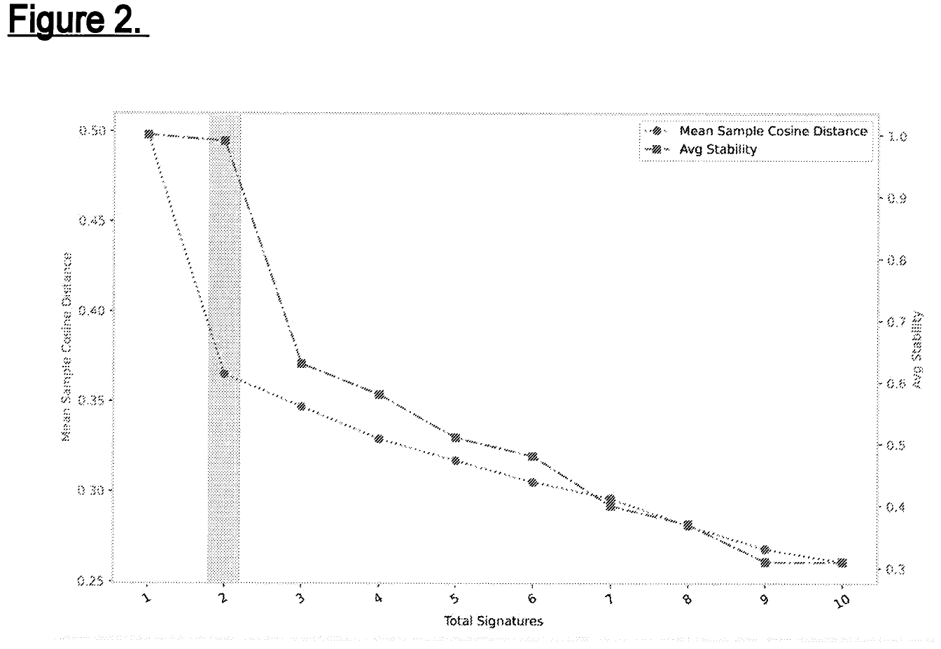

FIG. 3. Genomic and epigenomic samples. De novo signatures and optimal decomposition using COSMIC SBS signatures. HRD+. 64 Breast cancer samples (TUTT samples). Selected samples with >10 somatic SNVs. Identified 1 de novo signature using NMF. SBS3 contribution is 45.76%. HRD− 192 samples (BreastTN, Lung, CRC) without deleterious mutations in HRR pathway. Selected samples with >10 somatic SNVs. Identified 2 de novo signatures using NMF SBS3 not found.

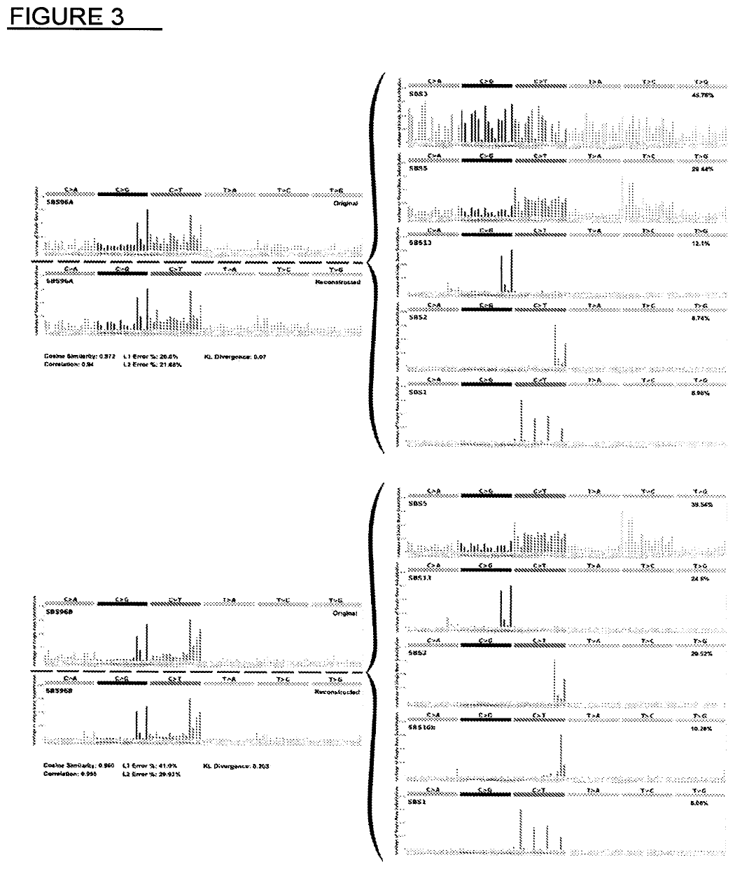

FIG. 4. Genomic samples. De novo signatures, optimal decomposition using COSMIC SBS signatures HRD+: Selected samples with cnv_tf & max_maf>10%. Selected samples with >10 somatic SNVs. 140 Breast cancer samples. Extracted 2 de novo signature using NMF. SBS3not found. HRD−: Selected samples with cnv_tf & max_maf>10%. Selected samples with >10somatic SNVs. 140 Breast cancer samples. Extracted 2 de novo signature using NMF SBS3 not found

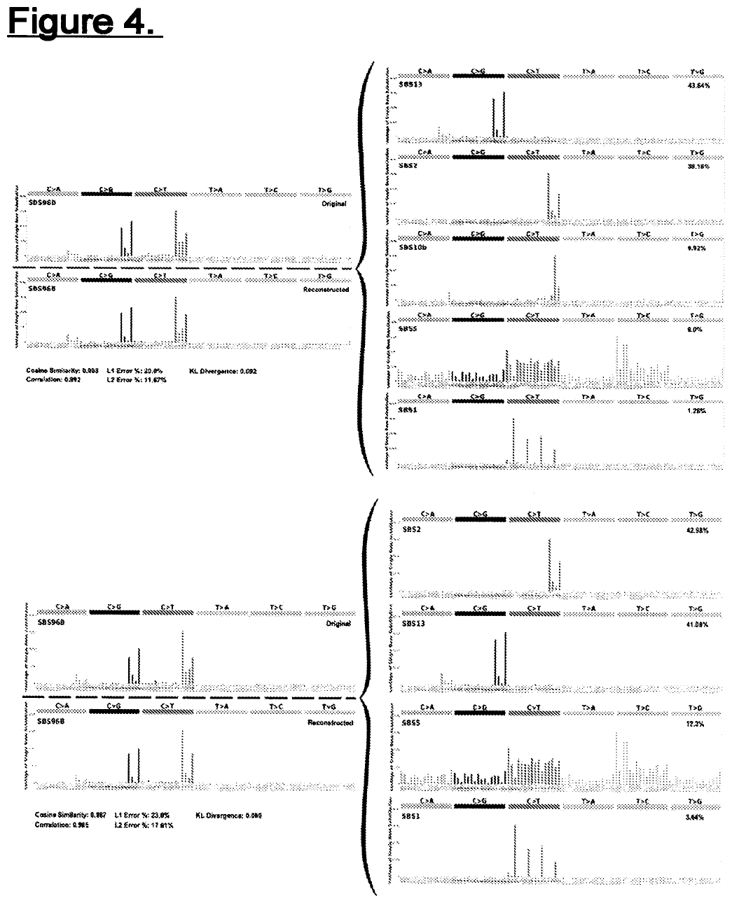

FIG. 5. Feature extraction and HRD classification (genomic and epigenomic samples). Mutational profile of every sample is decomposed into the previously found de novo signatures (positive and negative) using the NNLS. Use the normalized signature weights as features to train the ML classification algorithm. Here the HRD score is the probability that the sample's SBS profile is derived from an HRD+ sample.

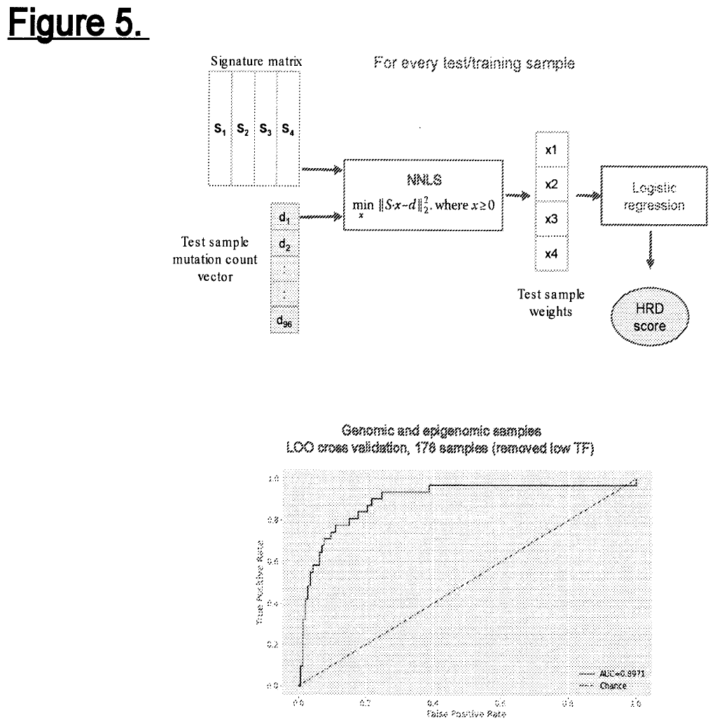

FIG. 6. Test the classifier on genomic data. The classification algorithm successfully identifies a subset of genomic samples enriched with Signature 3. Selected 25 Breast Cancer genomic samples with SBS3-based score>0.6. Analyze the mutational profiles of the selected genomic samples as described previously

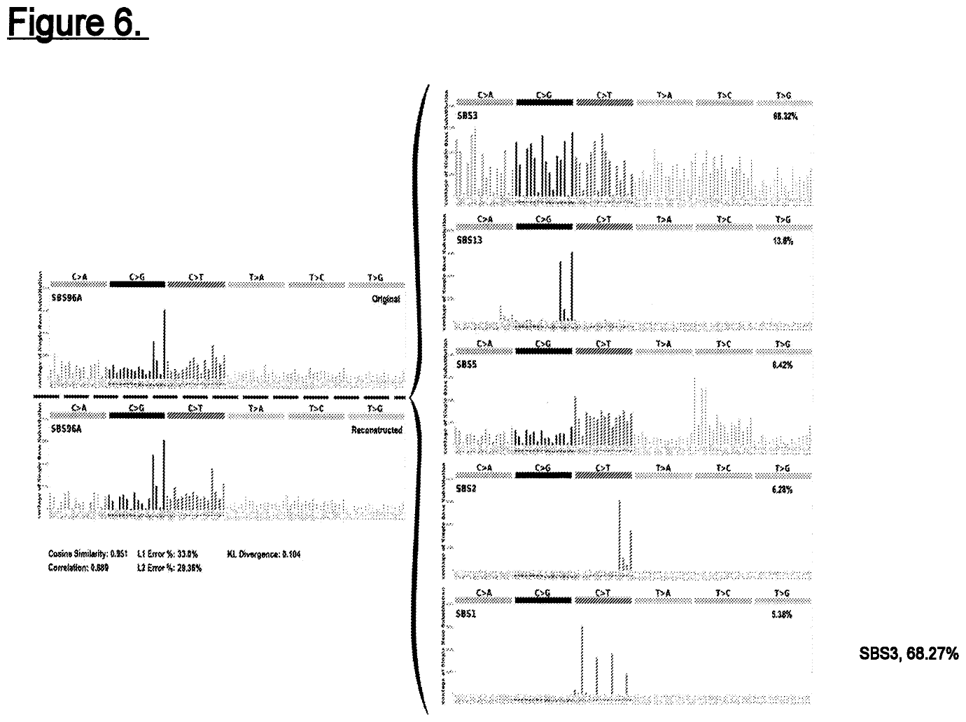

FIG. 7. Ensemble model for HRD score. SBS: Likelihood that the single base substitution profile of the sample is derived from an HRD+ sample. LST: Large-scale state transitions counts the number of chromosomal breaks between adjacent regions of at least a certain size. LOH: Loss of Heterozygosity score counts LoH segments with at least a certain size. TAI: Number of Telomeric Allelic Imbalances counts the number of regions with allelic imbalance which extend to the sub telomere but do not cross the centromere. CNV: Probability of a sample being HRD+ based on copy number signatures derived from copy number profiles through mixture model fitting and NMF transformation. METH: Probability of sample being HRD+ based on normalized counts of hyper partition molecules overlapping targeted regions in the methylation panel.

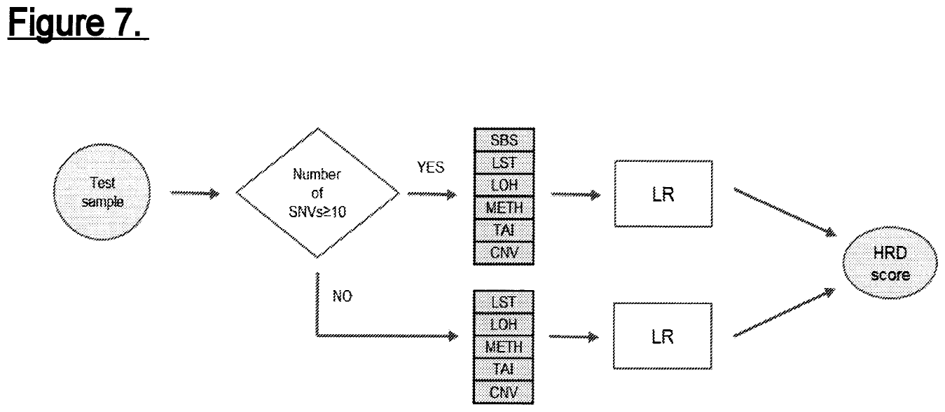

FIG. 8. Principal component analysis. The PCA plot shows mutational signatures are distinct with a higher degree of similarity between BSS96Bn and SBS96Bp as indicated by their closer proximity along PC1. This indicates that the first two principal components (PC1 and PC2) effectively capture the variation in mutation frequencies, highlighting the unique mutational patterns of each signature.

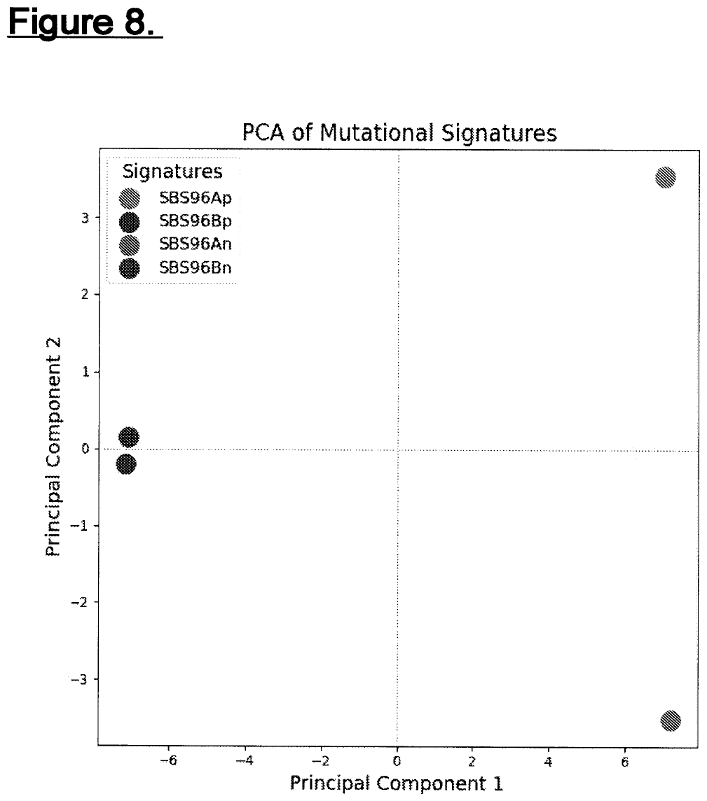

FIG. 9. Top 10 mutation types contributing most to PC1 and PC2 and their respective loadings. The bar plots show the top 10 mutation types whose frequencies vary the most across the signatures, as indicated by their high loadings (contribute most to the overall variance) on PC1 and PC2.

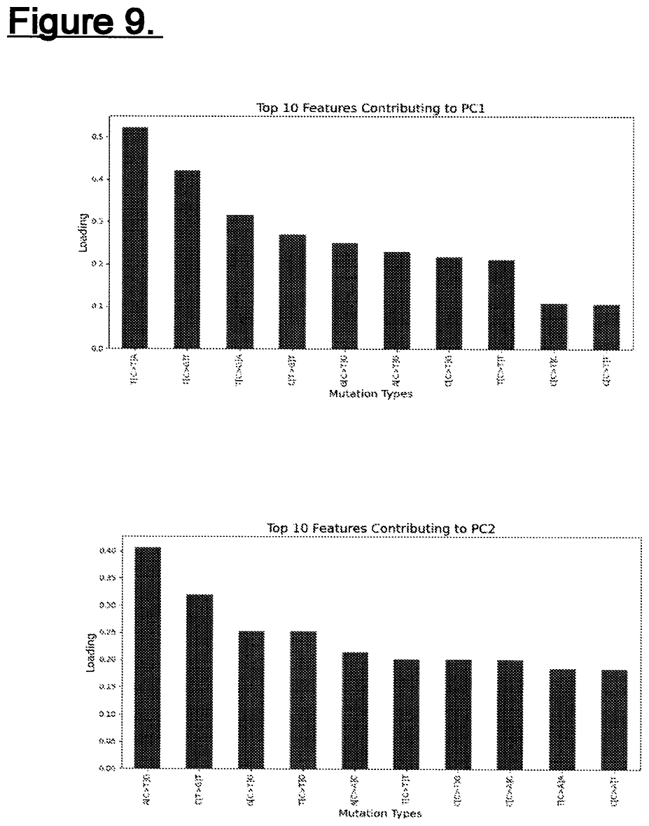

FIG. 10. The heatmap shows the frequencies of mutation types across different signatures. Variant T[C>T]A exhibits high frequencies in both SBS96Bp and SBS96Bn, C[T>G]T shows the highest frequency in SBS96Ap, while variant A[C>T]G has the highest frequency in SBS96An.

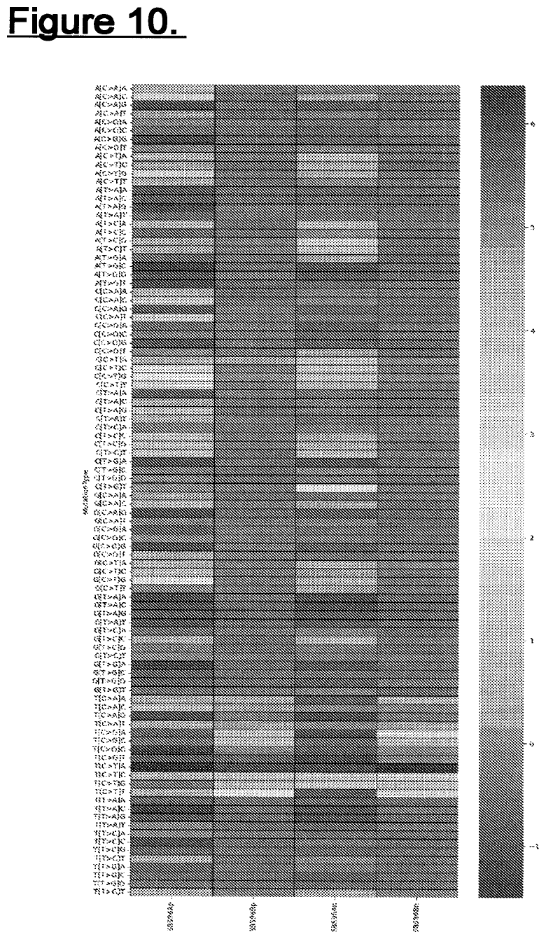

### SUMMARYOF THE INVENTION

Described herein is a method, including: determining context of at least one mutated position from of a plurality of nucleic acids each obtained from a plurality of samples, creating at least one matrix including a sample and the at least mutation context, processing the at least one matrix to generate one or more mutational signatures, determining at least one metric for each of the plurality of samples. In other embodiments, the at least one metric trains a classification algorithm. In other embodiments, the trained classification algorithm calculates a probability of a test sample being HRD positive or HRD negative. In other embodiments, the at least one matrix comprises non-negative matrix factorization. In other embodiments, the at least one metric comprises a feature vector including non-negative weights (NNW) determined using non-negative lease squares (NNLS).

Also described herein is a method, including determining context of at least one mutated position from of a plurality of nucleic acids each obtained from a plurality of samples, creating at least one matrix including a sample and the at least mutation context, processing the at least one matrix to generate one or more mutational signatures, determining at least one metric for each of the plurality of samples, training a classification algorithm with the at least one metric, calculating a probability of a test sample being HRD positive or HRD negative using the trained classification algorithm.

Also described herein is a method, including, determining context of at least one mutated position from of a plurality of nucleic acids each obtained from a plurality of HRD positive or HRD negative samples, wherein the context comprises one nucleotide upstream and one nucleotide downstream, creating at least one matrix including a sample and the at least mutation context, processing the at least one matrix to generate one or more mutational signatures using non-negative matrix factorization, determining at least one metric for each of the plurality of samples, wherein the at least one metric comprises a feature vector including non-negative weights (NNW) determined using non-negative lease squares (NNLS), training a classification algorithm with the at least one metric, calculating a probability of a test sample being HRD positive or HRD negative and using the trained classification algorithm. In various embodiment, the method generate a signature including about 5-10, 10-20, 20-30, 30-40, 40-50, 50 or more trinucleotides.

Described herein is a method, including determining, by computing system and implementing a predictive model, individual probabilities of a homologous recombination repair deficiency being present in individual samples in a plurality of samples, and determining, by the computing system and based on the individual probabilities, a probability to indicate a homologous recombination repair deficiency being present with respect to a given subject. In other embodiments, the computing system, a responsiveness to treatment with respect to a group of subjects, wherein cancer is detected in the group of subjects and the treatment is provided to treat the cancer, and determining, by the computing system, the plurality of samples that correspond to subjects having a homologous recombination repair deficiency based on the responsiveness of a portion of the group of subjects to the treatment. In other embodiments, the treatment is a poly adenosine diphosphate (ADP) ribose polymerase (PARP) inhibitor.

Described herein is a method, including determining, by computing system a probability of a homologous recombination repair deficiency being present in a human subject, wherein the determination is made using a single base substitution (SBS) signature. In various embodiments, the SBS signature is determined by one of the methods described herein. In various embodiments, the SBS signature includes about 5-10, 10-20, 20-30, 30-40, 40-50, 50 or more trinucleotides. In another embodiment, a determination of treatment is used using the SBS signature. In various embodiments, the determination is made using a database. In various embodiments, the determined treatment is administere method of any aforementioned claim, wherein the at least one metric trains a classification algorithm. In various embodiments, the training comprises a linear classifier, neutral network, decision tree, kernel estimation, support vector machine. In various embodiments, the trained classification algorithm calculates a probability of a test sample being HRD positive or HRD negative. In various embodiments, processing the at least one matrix comprises non-negative matrix factorization. In various embodiments, the at least one metric comprises a feature vector comprising non-negative weights (NNW) determined using non-negative lease squares (NNLS). In various embodiments, determining the context of at least one mutated position comprises identifying at least one nucleotide upstream and one nucleotide downstream of the mutated position. In various embodiments, creating at least one matrix comprises one or more rows and one or more columns.

In various embodiments, the method includes, creating at least one matrix comprises a row comprising one or more training samples and columns comprising a single base mutation in the determined context. In various embodiments, the method includes obtaining a sample from a human subject. In various embodiments, the sample comprises cell free DNA (cfDNA). In various embodiments, the method includes selecting a treatment based on the determination of at least one metric. In various embodiments, the treatment is a poly adenosine diphosphate (ADP) ribose polymerase (PARP) inhibitor. In various embodiments, the method includes administration of the treatment to a human subject.

In various embodiment, the method includes an ensemble score for HRD including one or more of SBS: Likelihood that the single base substitution profile of the sample is derived from an HRD+ sample, LST: Large-scale state transitions counts the number of chromosomal breaks between adjacent regions of at least a certain size, LOH: Loss of Heterozygosity score counts LoH segments with at least a certain size, TAI: Number of Telomeric Allelic Imbalances counts the number of regions with allelic imbalance which extend to the sub-telomere but do not cross the centromere, CNV: Probability of a sample being HRD+ based on copy number signatures derived from copy number profiles through mixture model fitting and NMF transformation, and METH: Probability of sample being HRD+ based on normalized counts of hyper partition molecules overlapping targeted regions in the methylation panel.

The method includes generating, by a computing system, the trained predictive model to determine a probability of a homologous recombination repair deficiency being present in one or more additional subjects, the predictive model including a plurality of variables and a plurality of weights with individual weights of the plurality of weights corresponding to individual variables of the plurality of variables, where an individual variable of the plurality of variables corresponds to an individual classification region of the subset of the plurality of classification regions and an individual weight that corresponds to the individual variable indicates a likelihood of the individual classification region indicating a homologous recombination repair deficiency.

In one or more examples, the method may include analyzing, by the computing system, the subset of training sequence representations to determine additional quantitative measures derived from a subset of training sequence reads.

In various examples, the method may include determining, by the computing system and implementing the predictive model, individual probabilities of a homologous recombination repair deficiency being present in individual samples of the plurality of samples based on the normalized quantitative measures corresponding to the individual samples, and determining, by the computing system and based on the individual probabilities, a threshold probability to indicate a homologous recombination repair deficiency being present with respect to a given subject.

In addition, the method may include determining, by the computing system, a responsiveness to treatment with respect to a group of subjects, where cancer is detected in the group of subjects and the treatment is provided to treat the cancer, and determining, by the computing system, the plurality of samples that correspond to subjects having a homologous recombination repair deficiency based on the responsiveness of a portion of the group of subjects to the treatment being at least a threshold level of responsiveness.

Further, the method may include analyzing, by the computing system, additional sequence reads derived from samples of a group of subjects in which cancer is detected to determine whether one or more genomic mutations are present with respect to one or more genomic regions, where the one or more genomic mutations correspond to homologous recombination repair path ways, and determining, by the computing system, the plurality of samples used to produce the training sequence representations by identifying a portion of the samples derived from the group of subjects in which the one or more genomic mutations are present.

In one or more additional examples, the method may include implementing, by the computing system, the trained predictive model to determine a probability of a homologous recombination repair deficiency being present in a plurality of additional samples, the plurality of additional samples being derived from additional subjects with a first form of cancer being detected in a first portion of the additional subjects and a second form of cancer being detected in a second portion of the additional subjects.

In one or more further examples, the method may include implementing, by the computing system, the predictive model to determine a probability of a homologous recombination repair deficiency being present in a plurality of additional samples, the plurality of additional samples being derived from additional subjects in which a single form of cancer is present.

In one or more examples, the method may include analyzing, by the computing system, the subset of training sequence reads to determine a group of training sequence reads that correspond to a plurality of genomic regions associated with homologous recombination repair pathways, and determining, by the computing system, one or more additional quantitative measures based on a number of the group of training sequence representations that correspond to at least a portion of the plurality of genomic regions,

The method may also include determining, by the computing system, tumor fraction estimates for a number of samples, the number of samples corresponding to subjects in which cancer is detected, analyzing, by the computing system, the tumor fraction estimates with respect to a threshold tumor fraction estimate, and determining, by the computing system, the plurality of samples used to derive the training sequence reads based on identifying at least a portion of the number of samples having a tumor fraction estimate corresponding to at least the threshold tumor fraction estimate.

In various examples, the method may include obtaining, by the computing system, testing sequence data from an additional subject that is not included in the plurality of subjects, the testing sequence data including testing sequencing representations derived from a sample of the additional subject, individual testing sequencing representations including a nucleotide sequence corresponding to a fragment of a nucleic acid included in the additional sample and individual testing sequencing reads corresponding to molecules having the threshold amount of methylated cytosines included in regions of the nucleotide, and determining, using the predictive model and the additional sequence data, a probability of a homologous recombination repair deficiency being present in the additional subject.

Further, the method may include combining a plurality of nucleic acids derived from at least one of blood or tissue of a subject with a solution including an amount of methyl binding domain (MBD) proteins to produce a nucleic acid-MBD protein solution, and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least a threshold cytosine-guanine content. Other technical features may be readily apparent to one skilled in the art from the following figures, descriptions, and claims.

In one or more additional examples, the treatment may include a poly adenosine diphosphate (ADP) ribose polymerase (PARP) inhibitor.

The method may also include analyzing, by the computing system, differences between the one or more additional quantitative measures and the one or more further quantitative measures to determine one or more additional variables for the predictive model.

In addition, the method may include analyzing, by the computing system, the testing sequencing reads to determine first additional quantitative measures that correspond to the individual classification regions of the plurality of classification regions, analyzing, by the computing system, the testing sequencing reads to determine second additional quantitative measures derived from the testing sequencing reads that correspond to individual control regions of a plurality of control regions, the individual control regions of the plurality of control regions having the threshold amount of methylated cytosines in subjects in which cancer is detected and in further subjects in which cancer is not detected, determining, by the computing system, additional normalized quantitative measures that correspond to the subset of the plurality of classification regions, where an individual additional normalized quantitative measure is determined according to the first additional quantitative measures and the second additional quantitative measures, and generating, by the computing system, an input vector that includes the normalized quantitative measures, where the predictive model uses the input vector to determine the probability of a homologous recombination repair deficiency being present in the additional subject.

In at least some examples, the method may include determining that a first nucleic acid fraction is associated with a first partition of a plurality of partitions of nucleic acids, the first partition corresponding to a first range of binding energies to MBD proteins, causing a first molecular barcode to attach to nucleic acids of the first nucleic acid fraction, the first molecular barcode being associated with the first partition, determining that a second nucleic acid fraction is associated with a second partition of the plurality of partitions of nucleic acids, the second partition corresponding to a second range of binding energies to MBD proteins different from the first range of binding energies to MBD proteins, and causing a second molecular barcode to attach to nucleic acids of the second nucleic acid fraction, the second molecular barcode being associated with the second partition.

In various examples, the method may include combining at least a portion of the number of nucleic acid fractions with an amount of restriction enzyme that cleaves molecules with one or more unmethylated cytosines to produce at least a portion of the plurality of samples used to produce the training sequence representations, where the threshold amount of methylated cytosines corresponds to a minimum frequency of methylated cytosines within a region having at least the threshold cytosine-guanine content.

The method may also include combining at least a portion of the number of nucleic acid fractions with an amount of a restriction enzyme that cleaves molecules with one or more methylated cytosines to produce at least a portion of the plurality of samples used to produce the training sequence representations, where the threshold amount of unmethylated cytosines corresponds to a maximum frequency of methylated cytosines that are not cleaved within a region having at least the threshold cytosine-guanine content. Other technical features may be readily apparent to one skilled in the art from the following figures, descriptions, and claims.

The method of any aforementioned claim, wherein the training comprises a linear classifier, neutral network, decision tree, kernel estimation, support vector machine. The method of any aforementioned claim, wherein the trained classification algorithm calculates a probability of a test sample being HRD positive or HRD negative.

In various embodiments, the method includes processing the at least one matrix comprises non-negative matrix factorization. In various embodiments, the method includes, the at least one metric comprises a feature vector comprising non-negative weights (NNW) determined using non-negative lease squares (NNLS). In various embodiments, the method includes determining the context of at least one mutated position comprises identifying at least one nucleotide upstream and one nucleotide downstream of the mutated position. In various embodiments, the method includes creating at least one matrix comprises one or more rows and one or more columns. In various embodiments, the method includes creating at least one matrix comprises a row comprising one or more training samples and columns comprising a single base mutation in the determined context. In various embodiments, the method includes obtaining a sample from a human subject. In various embodiments, the method includes the sample comprises cell free DNA (cfDNA). In various embodiments, the method includes selecting a treatment based on the determination of at least one metric. In various embodiments, the method includes the treatment is a poly adenosine diphosphate (ADP) ribose polymerase (PARP) inhibitor. In various embodiments, the method includes administration of the treatment to a human subject.

In one aspect, a computing apparatus includes a processor. The computing apparatus also includes a memory storing instructions that, when executed by the processor, configure the apparatus to obtain training sequence data including training sequence representations derived from a plurality of samples, individual training sequence representations including a nucleotide sequence corresponding to a fragment of a nucleic acid included in a sample of a plurality of samples and individual samples of the plurality of samples corresponding to a subject classified as having a homologous recombination repair deficiency, determine a subset of the training sequence representations that correspond to nucleic acids having at least a threshold amount of methylated cytosines in one or more regions of the nucleotide sequence, analyze the subset of training sequence representations to determine quantitative measures derived from the subset of the training sequence representations, individual quantitative measures corresponding to a classification region of a plurality of classification regions of a reference genome, individual classification regions of the plurality of classification regions having the threshold amount of methylated cytosines in subjects in which cancer is detected, analyze, using one or more computational techniques, the quantitative measures of the plurality of classification regions to determine a subset of the plurality of classification regions having at least a threshold likelihood of indicating a homology directed repair deficiency, and generate a predictive model to determine a probability of a homologous recombination repair deficiency being present in one or more additional subjects, the predictive model including a plurality of variables and a plurality of weights with individual weights of the plurality of weights corresponding to individual variables of the plurality of variables, where an individual variable of the plurality of variables corresponds to an individual classification region of the subset of the plurality of classification regions and an individual weight that corresponds to the individual variable indicates a likelihood of the individual classification region indicating a homologous recombination repair deficiency.

The computing apparatus may also include additional instructions that, when executed by the processor, configure the apparatus to analyze the subset of training sequence representations to determine additional quantitative measures derived from the subset of the training sequence reads, individual quantitative measures corresponding to a control region of a plurality of control regions of a reference genome, individual control regions of the plurality of control regions having the threshold amount of methylated cytosines in subjects in which cancer is detected and in further subjects in which cancer is not detected, and determine normalized quantitative measures that correspond to the subset of the plurality of classification regions, where an individual normalized quantitative measure is determined according to the quantitative measure that corresponds to a classification region of the subset of the plurality of classification regions and the additional quantitative measures.

In addition, the computing apparatus may include additional instructions that, when executed by the processor, configure the apparatus to determine, by implementing the predictive model, individual probabilities of a homologous recombination repair deficiency being present in individual samples of the plurality of samples based on the normalized quantitative measures corresponding to the individual samples, and determine, by the computing system and based on the individual probabilities, a threshold probability to indicate a homologous recombination repair deficiency being present with respect to a given subject.

Further, the computing apparatus may include additional instructions that, when executed by the processor, configure the apparatus to determine a responsiveness to treatment with respect to a group of subjects, where cancer is detected in the group of subjects and the treatment is provided to treat the cancer, and determine the plurality of samples that correspond to subjects having a homologous recombination repair deficiency based on the responsiveness of a portion of the group of subjects to the treatment being at least a threshold level of responsiveness.

In one or more examples, the computing apparatus may include additional instructions that, when executed by the processor, configure the apparatus to analyze additional sequence reads derived from samples of a group of subjects in which cancer is detected to determine whether one or more genomic mutations are present with respect to one or more genomic regions, where the one or more genomic mutations correspond to homologous recombination repair pathways, and determine the plurality of samples used to produce the training sequence representations by identifying a portion of the samples derived from the group of subjects in which the one or more genomic mutations are present.

In various examples, the one or more computational techniques include implementing one or more logistic regression models with elastic regularization.

In at least some examples, the computing apparatus may include additional instructions that, when executed by the processor, configure the apparatus to implement the predictive model to determine a probability of a homologous recombination repair deficiency being present in a plurality of additional samples, the plurality of additional samples being derived from additional subjects with a first form of cancer being detected in a first portion of the additional subjects and a second form of cancer being detected in a second portion of the additional subjects.

In one or more additional examples, the computing apparatus may include additional instructions that, when executed by the processor, configure the apparatus to implement the predictive model to determine a probability of a homologous recombination repair deficiency being present in a plurality of additional samples, the plurality of additional samples being derived from additional subjects in which a single form of cancer is present.

In one or more further examples, the computing apparatus may include additional instructions that, when executed by the processor, configure the apparatus to analyze the subset of training sequence reads to determine a group of training sequence reads that correspond to a plurality of genomic regions associated with homologous recombination repair pathways, and determine one or more additional quantitative measures based on a number of the group of training sequence representations that correspond to at least a portion of the plurality of genomic regions,

In various examples, the plurality of classification regions have at least a threshold amount of cytosine-guanine content.

The computing apparatus may also include additional instructions that, when executed by the processor, configure the apparatus to determine tumor fraction estimates for a number of samples, the number of samples corresponding to subjects in which cancer is detected, analyze the tumor fraction estimates with respect to a threshold tumor fraction estimate, and determine, by the computing system, the plurality of samples used to derive the training sequence reads based on identifying at least a portion of the number of samples having a tumor fraction estimate corresponding to at least the threshold tumor fraction estimate.

Additionally, the computing apparatus may include additional instructions that, when executed by the processor, configure the apparatus to obtain testing sequence data from an additional subject that is not included in the plurality of subjects, the testing sequence data including testing sequencing representations derived from a sample of the additional subject, individual testing sequencing representations including a nucleotide sequence corresponding to a fragment of a nucleic acid included in the additional sample and individual testing sequencing reads corresponding to molecules having the threshold amount of methylated cytosines included in regions of the nucleotide, and determine, using the predictive model and the additional sequence data, a probability of a homologous recombination repair deficiency being present in the additional subject.

Further, the computing apparatus may include additional instructions that, when executed by the processor, configure the apparatus to analyze differences between the one or more additional quantitative measures and the one or more further quantitative measures to determine one or more additional variables for the predictive model.

In addition, the one or more non-transitory computer-readable storage media may include instructions that when executed by a computer, cause the computer to determine, by implementing the predictive model, individual probabilities of a homologous recombination repair deficiency being present in individual samples of the plurality of samples based on the normalized quantitative measures corresponding to the individual samples, and determine, based on the individual probabilities, a threshold probability to indicate a homologous recombination repair deficiency being present with respect to a given subject.

Further, the one or more non-transitory computer-readable storage media may include instructions that when executed by a computer, cause the computer to determine a responsiveness to treatment with respect to a group of subjects, where cancer is detected in the group of subjects and the treatment is provided to treat the cancer, and determine the plurality of samples that correspond to subjects having a homologous recombination repair deficiency based on the responsiveness of a portion of the group of subjects to the treatment being at least a threshold level of responsiveness.

In one or more examples, the one or more non-transitory computer-readable storage media may include instructions that when executed by a computer, cause the computer to analyze additional sequence reads derived from samples of a group of subjects in which cancer is detected to determine whether one or more genomic mutations are present with respect to one or more genomic regions, where the one or more genomic mutations correspond to homologous recombination repair pathways, and determine the plurality of samples used to produce the training sequence representations by identifying a portion of the samples derived from the group of subjects in which the one or more genomic mutations are present.

The one or more computational techniques include implementing one or more logistic regression models with elastic regularization.

In one or more additional examples, the one or more non-transitory computer-readable storage media may include instructions that when executed by a computer, cause the computer to implement the predictive model to determine a probability of a homologous recombination repair deficiency being present in a plurality of additional samples, the plurality of additional samples being derived from additional subjects with a first form of cancer being detected in a first portion of the additional subjects and a second form of cancer being detected in a second portion of the additional subjects.

In one or more further examples, the one or more non-transitory computer-readable storage media may include instructions that when executed by a computer, cause the computer to implement the predictive model to determine a probability of a homologous recombination repair deficiency being present in a plurality of additional samples, the plurality of additional samples being derived from additional subjects in which a single form of cancer is present.

In at least some examples the one or more non-transitory computer-readable storage media may include instructions that when executed by a computer, cause the computer to analyze the subset of training sequence reads to determine a group of training sequence reads that correspond to a plurality of genomic regions associated with homologous recombination repair pathways, and determine one or more additional quantitative measures based on a number of the group of training sequence representations that correspond to at least a portion of the plurality of genomic regions,

In various examples, the plurality of classification regions have at least a threshold amount of cytosine-guanine content.

The one or more non-transitory computer-readable storage media may also include instructions that when executed by a computer, cause the computer to determine tumor fraction estimates for a number of samples, the number of samples corresponding to subjects in which cancer is detected, analyze the tumor fraction estimates with respect to a threshold tumor fraction estimate, and determine the plurality of samples used to derive the training sequence reads based on identifying at least a portion of the number of samples having a tumor fraction estimate corresponding to at least the threshold tumor fraction estimate.

The one or more non-transitory computer-readable storage media may also include instructions that when executed by a computer, cause the computer to determine an additional subset of the training sequence representations that correspond to additional nucleic acids having less than an additional threshold amount of methylation, analyze, by the computing system, the additional subset of the training sequence reads to determine an additional group of training sequence representations that correspond to the plurality of genomic regions associated with the homologous recombination repair pathways, determine one or more further quantitative measures based on an additional number of the additional group of training sequence representations that correspond to at least a portion of the plurality of genomic regions.

Additionally, the one or more non-transitory computer-readable storage media may include instructions that when executed by a computer, cause the computer to analyze differences between the one or more additional quantitative measures and the one or more further quantitative measures to determine one or more additional variables for the predictive model.

Further, the one or more non-transitory computer-readable storage media may include instructions that when executed by a computer, cause the computer to analyze the testing sequencing reads to determine first additional quantitative measures, analyze, by the computing system, the testing sequencing reads to determine second additional quantitative measures derived from the testing sequencing reads, comparing to subjects in which cancer is detected and in further subjects in which cancer is not detected, determine additional normalized quantitative measures that correspond to the subset of the plurality of classification regions, where an individual additional normalized quantitative measure is determined according to the first additional quantitative measures and the second additional quantitative measures, and generate an input vector that includes the normalized quantitative measures, where the predictive model uses the input vector to determine the probability of a homologous recombination repair deficiency being present in the additional subject.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth.

It is also to be understood that the terminology used herein is for the purpose of describing particular implementations only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain implementations, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

### DETAILED DESCRIPTION

Cell survival and replication rely heavily on the vital capacity to accurately replicate and repair DNA. Multiple DNA repair mechanisms exist and double-strand breaks (DSBs) can be repaired by 2 distinct processes in the cell. This includes Homologous Recombination Repair (HRR) pathway which provides DNA repair with high fidelity. The other is Non-Homologous End Joining (NHEJ) pathway which is error-prone. The HRR pathway can be lost in some cancers such as breast, ovarian, endometrial, pancreatic, and prostate.

Cancers with HRD (HRD+) are sensitive to targeted inhibition of PARP enzymes since PARP is a key component of the NHEJ pathways. The identification of patients with HRD biomarkers enables the identification of individuals who are likely to benefit from PARPi therapies.

Cancer is usually caused by the accumulation of mutations within genes of an individual's cells, at least some of which result in improperly regulated cell division. Such mutations can include single nucleotide variations (SNVs), gene fusions, insertions, deletions, transversions, translocations, and inversions. These mutations can also include copy number variations that correspond to an increase or a decrease in the number of copies of a gene within a tumor genome relative to an individual's noncancerous cells. An extent of mutations present in cell-free nucleic acids and an amount of mutated cell-free nucleic acids of a sample can be used as biomarkers to determine tumor progression, predict patient outcome, and refine treatment choices. In various examples, the extent of mutations present in cell-free nucleic acids can be indicated by tumor cells copy number and tumor fraction for a given sample.

Of particular interest in cancer is homologous recombination deficiency (HRD) referes to a specific type of genetic instability or impairment in the DNA repair process that affects cancer cells. HRD is characterized by defects or alterations in the DNA repair pathway known as homologus recombination, which is responsible for repairing double-stranded DNA breaks. Knowing the HRD status of a patient's tumor provides valuable information for diagnosis, prognosis, and treatment selection. It helps to optimize cancer management and improve patient outcomes. The common way to detect HRD in tumor tissue is to examine genomic landscape of the tumor for large-scale genomic rearrangements, such as loss of heterozygosity (LOH), large-scale state transtitions (LST), and telomeric allelic imbalance (TAI).

However, a reliable and accurate calculation of these scores in targeted cfDNA samples presents a challenge, especially for samples with low tumor fraction. Mutational signatures provide a comprehensive view of the specific types of single base substitutions (SBS) observed at different positions in the genome. Existing techniques focus on somatic CNVs are usually manifested through Loss of heterozygosity (LOH): loss of a single allele. Large-scale transitions (LST): chromosomal breaks between adjacent regions of at least 10 Mb. Telomeric allelic imbalance (TAI): regions of copy number loss extending to telomeres. However, given the scope of mutational SBS signatures, this approach provide insights into the underlying causes of DNA damage, with some reported to be associated with defective homologous recombination DNA repair. To date, mutational SBS signatures have not applicable to cfDNA, given the small size of analyte and profound scarcity compared to existing efforts which rely on whole genome sequencing (WGS).

The methods and systems described herein are directed to determining subjects having homologous recombination repair deficiencies by analyzing signatures obtained from samples including cell-free nucleic acids of the subjects. In addition, the signature data can correspond to genomic regions that have been previously identified as having one or more mutations present in individuals in which cancer is present. The analysis of signatures data, including de novo signatures obtained via the methods descried herein, to determine subjects in which homologous recombination repair deficiencies are present improves the accuracy of the detection of homologous recombination repair deficiencies in relation to the accuracy achieved using existing techniques.

In one or more implementations, one or more computational models can be generated that determine the status of subjects with respect to homologous recombination repair deficiencies. The one or more computational models can implement at least one of one or more machine learning techniques or one or more statistical techniques to determine the status of subjects with respect to homologous recombination repair deficiencies. In various examples, the one or more computational models can analyze sequencing data that corresponds to samples obtained from subjects to determine the status of the subjects with respect to homologous recombination repair deficiencies. Genomic regions can exhibit one or more mutations in individuals in which one or more forms of cancer are present and/or in individuals in which homologous recombination repair deficiencies are present. The number of genomic regions can also include differentially methylated regions in individuals in which homologous recombination repair deficiencies are present.

In various examples, the sequence representations provided to the predictive computational model during the training process or after the training process have at least a threshold amount of methylation of cytosines in classification regions. The sequence representations that satisfy the methylation levels can be produced, at least in party, using one or more molecule separation processes. The molecule separation processes can include combining a plurality of nucleic acids derived from at least one of blood or tissue of a subject with a solution including an amount of methyl binding domain (MBD) proteins to produce a nucleic acid-MBD protein solution. A plurality of washes can then be performed of the nucleic acid-MBD protein solution with a salt solution to produce a number of nucleic acid fractions. Individual nucleic acid fractions can have a threshold number of molecules with a methylated cytosine in regions of the plurality of nucleic acids having at least the threshold cytosine-guanine content. In one or more illustrative examples, a wash of the plurality of washes can be performed with a solution having a concentration of sodium chloride (NaCl) and can produce a nucleic acid fraction of the number of nucleic acid fractions having a range of binding energies to MBD proteins.

In one or more examples, a first nucleic acid fraction can be determined is associated with a first partition of a plurality of partitions of nucleic acids. The first partition corresponding to a first range of binding energies to MBD proteins. Further, a first molecular barcode can be attached to nucleic acids of the first nucleic acid fraction. The first molecular barcode can be associated with the first partition. In addition, a second nucleic acid fraction can be determined that is associated with a second partition of the plurality of partitions of nucleic acids. The second partition can correspond to a second range of binding energies to MBD proteins different from the first range of binding energies to MBD proteins. A second molecular barcode can be attached to nucleic acids of the second nucleic acid fraction. The second molecular barcode being associated with the second partition.

### SAMPLES

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some implementations, the sample volume of body fluid taken from a subject depends on the desired read depth for sequenced regions. Example volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled blood can be between about 5 ml to about 20 ml.

The sample can comprise various amounts of nucleic acid. The amount of nucleic acid in a given sample can be equated with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some implementations, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations). In some implementations of the present disclosure, cell free nucleic acids in a subject may derive from a tumor. For example, cell-free DNA isolated from a subject can comprise ctDNA.

Example amounts of cell-free nucleic acids in a sample before amplification typically range from about 1 femtogram (fg) to about 1 microgram (μg), e.g., about 1 picogram (pg) to about 200nanogram (ng), about 1 ng to about 100 ng, about 10 ng to about 1000 ng. In some implementations, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200ng of cell-free nucleic acid molecules. In certain implementations, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some implementations, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain implementations, cell-free nucleic acids are from about 160 to about 180 nucleotides in length, or from about 320 to about 360 nucleotides in length, or from about 440 to about 480 nucleotides in length.

In some implementations, cell-free nucleic acids are isolated from bodily fluids through a partitioning step in which cell-free nucleic acids, as found in solution, are separated from intact cells and other non-soluble components of the bodily fluid. In some of these implementations, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain implementations, additional clean up steps are used, such as silica-based columns to remove contaminants or salts. Non-specific bulk carrier nucleic acids, for example, are optionally added throughout the reaction to optimize certain aspects of the example procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

### NUCLEIC ACID TAGS

In certain implementations, tags providing molecular identifiers or barcodes are incorporated into or otherwise joined to adapters by chemical synthesis, ligation, or overlap extension PCR, among other methods. In some implementations, the assignment of unique or non-unique identifiers, or molecular barcodes in reactions follows methods and utilizes systems described in, for example, U.S. patent applications Ser. No. 20/010,053519, 20030152490, 20110160078, and U.S. Pat. Nos. 6,582,908, 7,537,898, and 9,598,731, which are each incorporated by reference.

Tags are linked (e.g., ligated) to sample nucleic acids randomly or non-randomly. In some implementations, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some implementations, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain implementations, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique or non-unique.

One example format uses from about 2 to about 1,000,000 different tags, or from about 5to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some implementations, identifiers are predetermined, random, or semi-random sequence oligonucleotides. In other implementations, a plurality of barcodes may be used such that barcodes are not necessarily unique to one another in the plurality. In these implementations, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

### NUCLEIC ACID AMPLIFICATION

Sample nucleic acids flanked by adapters are typically amplified by PCR and other amplification methods using nucleic acid primers binding to primer binding sites in adapters flanking a DNA molecule to be amplified. In some implementations, amplification methods involve cycles of extension, denaturation and annealing resulting from thermocycling, or can be isothermal as, for example, in transcription mediated amplification. Other example amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce sample indexes/tags to a nucleic acid molecule using conventional nucleic acid amplification methods. The amplifications are typically conducted in one or more reaction mixtures. In some implementations, molecular tags and sample indexes/tags are introduced prior to and/or after sequence capturing steps are performed. In some implementations, only the molecular tags are introduced prior to probe capturing and the sample indexes/tags are introduced after sequence capturing steps are performed. In certain implementations, both the molecular tags and the sample indexes/tags are introduced prior to performing probe-based capturing steps. In some implementations, the sample indexes/tags are introduced after sequence capturing steps (i.e., enrichment of nucleic acids) are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some implementations, the amplicons have a size of about 300 nt. In some implementations, the amplicons have a size of about 500 nt.

### NUCLEIC ACID ENRICHMENT

In some implementations, sequences are enriched prior to sequencing the nucleic acids. Enrichment is optionally performed for specific target regions or nonspecifically (“target sequences”). In some implementations, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some implementations, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain implementations, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

### NUCLEIC ACID SEQUENCING

After extraction and isolation of cfDNA from samples, the cfDNA may be sequenced at steps 103 and 104. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, primer walking, sequencing using PacBio, SOLID, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some implementations, cell-free polynucleotides are sequenced with at least about 1000, 2000, 3000, 4000, 5000, 6000, 7000, 8000, 9000, 10000, 50000, or 100,000 sequencing reactions. In other implementations, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some implementations, data analysis is performed on at least about 1000, 2000, 3000, 4000, 5000, 6000, 7000, 8000, 9000, 10000, 50000, or 100,000 sequencing reactions. In other implementations, data analysis may be performed on less than about 1000, 2000, 3000, 4000, 5000, 6000, 7000, 8000, 9000, 10000, 50000, or 100,000 sequencing reactions. An example read depth is from about 1000to about 50000 reads per locus (base position).

In some implementations, a nucleic acid population is prepared for sequencing by enzymatically forming blunt-ends on double-stranded nucleic acids with single-stranded overhangs at one or both ends. In these implementations, the population is typically treated with an enzyme having a 5′-3′ DNA polymerase activity and a 3′-5′ exonuclease activity in the presence of the nucleotides (e.g., A, C, G and T or U). Example enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some implementations, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some implementations, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a template/parent nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence may be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17:95-115 (2016), Liu et al., J. of Biomedicine and Biotechnology, Volume 2012, Article ID 251364:1-11 (2012), Voelkerding et al., Clinical Chem., 55:641-658 (2009), MacLean et al., Nature Rev. Microbiol., 7:287-296 (2009), Astier et al., J Am Chem Soc., 128 (5): 1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

### SEQUENCING PANEL

To improve the likelihood of detecting genomic regions of interest and optionally, tumor indicating mutations, the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions, for example, to detect a single cancer, a set of cancers, or all cancers. Alternatively, DNA may be sequenced by whole genome sequencing (WGS) or other unbiased sequencing method without the use of a sequencing panel. Examples of suitable panel and targets for use in panels can be found in the epigenetic targets described in U.S. provisional patent application 62/799,637, filed Jan. 31, 2019, which is incorporated by reference in its entirety. Other examples include Mutational Signatures found in Catalogue of Somatic Mutations in Cancer (COSMIC) available at https://cancer.sanger.ac.uk/signatures/sbs/.

In some aspects, a panel that targets a plurality of different genes or genomic regions (e.g., transcriptional factor binding regions, distal regulatory elements (DREs), repetitive elements, intron-exon junctions, transcriptional start sites (TSSs), and/or the like) is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some implementations, markers for a tissue of origin are tissue-specific epigenetic markers.

In some implementations, the one or more regions in the panel comprise one or more loci from one or a plurality of genes for detecting residual cancer after surgery. This detection can be earlier than is possible for existing methods of cancer detection. In some implementations, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some implementations, the methods described herein detect the response of patients to cancer therapy (particularly in high risk patients) earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A database may comprise information about regulatory elements or genomic regions in tumor samples. The information relating to the sequenced tumor samples may include the frequency of various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer I have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced, or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the mutant allele fraction for a given genetic variant. The mutant allele fraction may refer to the frequency at which mutant alleles occur in a given population of nucleic acids, such as a sample. Genetic variants at a low mutant allele fraction may have a relatively low frequency of presence in a sample. In some cases, the panel allows for detection of genetic variants at a mutant allele fraction of at least 0.0001%, 0.001%, 0.005%, 0.01%, 0.05%, 0.1%, or 0.5%. The panel can allow for detection of genetic variants at a mutant allele fraction of 0.001% or greater. The panel can allow for detection of genetic variants at a mutant allele fraction of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The locations comprising genomic regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy can be quantified using measures such as sensitivity and specificity, predictive values, likelihood ratios, the area under the ROC curve, Youden's index and/or diagnostic odds ratio.

Accuracy may be presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances, this may allow for more molecules within a biological to be analyzed thereby enabling lower frequency alleles to be detected.

In an implementation, after sequencing, sequence reads may be assigned a quality score. A quality score may be a representation of sequence reads that indicates whether those sequence reads may be useful in subsequent analysis based on a threshold. In some cases, some sequence reads are not of sufficient quality or length to perform a subsequent mapping step. Sequence reads with a quality score at least 90%, 95%, 99%, 99.9%, 99.99% or 99.999% may be filtered out of a data set of sequence reads. In other cases, sequence reads assigned a quality scored at least 90%, 95%, 99%, 99.9%, 99.99% or 99.999% may be filtered out of the data set. Sequence reads that meet a specified quality score threshold may be mapped to a reference genome. After mapping alignment, sequence reads may be assigned a mapping score. A mapping score may be a representation of sequence reads mapped back to the reference sequence indicating whether each position is or is not uniquely mappable. Sequence reads with a mapping score at least 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set.

TABLE 1
Frequency analysis of single base substitution signature

| Signature | Mutation Type | Frequency |
|---|---|---|
| SBS96Ap | C[T > G]T | 0.04389294 |
| SBS96Ap | G[C > T]G | 0.02744899 |
| SBS96Ap | C[C > T]G | 0.02728748 |
| SBS96Ap | C[C > T]T | 0.02341647 |
| SBS96Ap | A[C > A]C | 0.0224475 |
| SBS96Ap | A[C > T]G | 0.02155773 |
| SBS96Ap | C[C > T]C | 0.02135741 |
| SBS96Ap | C[C > A]C | 0.02056919 |
| SBS96Ap | C[C > A]T | 0.01876857 |
| SBS96Ap | C[T > C]T | 0.01755641 |
| SBS96Bp | T[C > T]A | 0.21758952 |
| SBS96Bp | T[C > G]T | 0.19587358 |
| SBS96Bp | T[C > G]A | 0.14293663 |
| SBS96Bp | T[C > T]T | 0.09293738 |
| SBS96Bp | T[C > T]G | 0.04884377 |
| SBS96Bp | T[C > G]C | 0.04746013 |
| SBS96Bp | T[C > T]C | 0.04258507 |
| SBS96Bp | T[C > A]A | 0.0292916 |
| SBS96Bp | T[C > A]C | 0.01947808 |
| SBS96Bp | T[C > A]T | 0.01887543 |
| SBS96An | A[C > T]G | 0.05596937 |
| SBS96An | G[C > T]G | 0.05360707 |
| SBS96An | C[C > T]G | 0.04978793 |
| SBS96An | C[T > G]T | 0.03707419 |
| SBS96An | T[C > T]G | 0.02497347 |
| SBS96An | A[T > C]G | 0.02448187 |
| SBS96An | A[C > T]A | 0.02177647 |
| SBS96An | T[C > T]C | 0.02115923 |
| SBS96An | C[C > T]A | 0.02102531 |
| SBS96An | A[T > C]T | 0.02081438 |
| SBS96Bn | T[C > T]A | 0.21324303 |
| SBS96Bn | T[C > G]T | 0.16390403 |
| SBS96Bn | T[C > G]A | 0.11553935 |
| SBS96Bn | T[C > T]T | 0.11319343 |
| SBS96Bn | T[C > T]C | 0.05411008 |
| SBS96Bn | T[C > T]G | 0.04493697 |
| SBS96Bn | T[C > A]A | 0.03704655 |
| SBS96Bn | T[C > G]C | 0.03222962 |
| SBS96Bn | T[C > A]T | 0.0217057 |
| SBS96Bn | T[C > A]C | 0.01488151 |

### PRECISION TREATMENTS

The precision diagnostics provided by the improved computer system 110 may result in precision treatment plans, which may be identified by the computer system 110 (and/or curated by health professionals). For example, one type of precision diagnostic and treatment may relate to genes in the homologous recombination repair (HRR) pathway.

Homologous recombination is a type of genetic recombination in which nucleotide sequences are exchanged between two similar or identical molecules of DNA. It is most widely used by cells to accurately repair harmful breaks that occur on both strands of DNA, known as double-strand breaks (DSB). HRR provides a mechanism for the error-free removal of damage present in DNA that has replicated (S and G2 phases), to eliminate chromosomal breaks before the cell division occurs. The primary model for how homologous recombination repairs double-strand breaks in DNA is homologous recombination repair pathway which mediates the double-strand break repair (DSBR) pathway and the synthesis-dependent strand annealing (SDSA) pathway. Germline and somatic deficiencies in homologous recombination genes have been strongly linked to breast, ovarian and prostate cancers.

The number and types of variant nucleotides in a sample can provide an indication of the amenability of the subject providing the sample to treatment, i.e., therapeutic intervention. For example, various poly ADP ribose polymerase (PARP) inhibitors have been shown to stop the growth of tumors from breast, ovarian and prostate cancers caused by hereditary mutations in the BRCA1 or BRCA2 genes. Some of these therapeutic agents may inhibit base excision repair (BER), which may compensate for the deficiency of HRR.

On the other hand, certain BRCA and HRR wildtype patients may not achieve clinical benefit from treatment with a PARP inhibitor. Furthermore, not all ovarian cancer patients with a BRCA mutation will respond to a PARP inhibitor. Moreover, different types of mutations may indicate different therapies. For example, somatic heterozygous deletions in HRR genes may indicate a different therapy than somatic homozygous deletions. Thus, the state of genetic material may influence therapy. In one example, a PARP inhibitor may be administered to an individual harboring a somatic homozygous deletion in a HRR gene, but not to an individual harboring a wildtype allele or somatic heterozygous deletions in the HRR gene.

In some implementations, a subject having HRD as determined by any of the methods disclosed may be administered a targeted therapy. The targeted therapy may comprise a PARP inhibitor. Examples of PARP inhibitors that may be administered include one or more of: VELIPARIB, OLAPARIB, TALAZOPARIB, RUCAPARIB, NIRAPARIB, PAMIPARIB, CEP 9722 (Cephalon), E7016 (Eisai), E7449 (Eisai, a PARP 1/2 and tankyrase 1/2 inhibitor), or 3-Aminobenzamide. In some implementations, the targeted therapy may comprise at least one base excision repair (BER) inhibitor. For example, OLAPARIB may inhibit BER. In certain implementations, the targeted therapy may comprise combination of a PARP inhibitor and radiotherapy. In an implementation, the combination of a PARP inhibitor and radiotherapy would permit the PARP inhibitor to lead to formation of double strand breaks from the single-strand breaks generated by the radiotherapy in tumor tissue (e.g., tissue with BRCA1/BRCA2 mutations). This combination can provide more powerful therapy per radiation dose.

### CUSTOMIZED THERAPIES AND RELATED ADMINISTRATIONS

In some implementations, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. In certain implementations, the therapy administered to a subject may comprise at least one chemotherapy drug. In some implementations, the chemotherapy drug may comprise alkylating agents (for example, but not limited to, Chlorambucil, Cyclophosphamide, Cisplatin and Carboplatin), nitrosourcas (for example, but not limited to, Carmustine and Lomustinc), anti-metabolites (for example, but not limited to, Fluorauracil, Methotrexate and Fludarabinc), plant alkaloids and natural products (for example, but not limited to, Vincristine, Paclitaxel and Topotecan), anti-tumor antibiotics (for example, but not limited to, Bleomycin, Doxorubicin and Mitoxantronc), hormonal agents (for example, but not limited to, Prednisone, Dexamethasone, Tamoxifen and Leuprolide) and biological response modifiers (for example, but not limited to, Herceptin and Avastin, Erbitux and Rituxan). In some implementations, the chemotherapy administered to a subject may comprise FOLFOX or FOLFIRI. Typically, therapies include at least one immunotherapy (or an immunotherapeutic agent). Immunotherapy refers generally to methods of enhancing an immune response against a given cancer type. In certain implementations, immunotherapy refers to methods of enhancing a T cell response against a tumor or cancer.

In some implementations, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain implementations, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain implementations, the inhibitory immune checkpoint molecule is CTLA4 or PD-1. In other implementations, the inhibitory immune checkpoint molecule is a ligand for PD-1, such as PD-L1 or PD-L2. In other implementations, the inhibitory immune checkpoint molecule is a ligand for CTLA4, such as CD80 or CD86. In other implementations, the inhibitory immune checkpoint molecule is lymphocyte activation gene 3(LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain implementations, the immunotherapy or immunotherapeutic agent is an antagonist of an inhibitory immune checkpoint molecule. In certain implementations, the inhibitory immune checkpoint molecule is PD-1. In certain implementations, the inhibitory immune checkpoint molecule is PD-L1. In certain implementations, the antagonist of the inhibitory immune checkpoint molecule is an antibody (e.g., a monoclonal antibody). In certain implementations, the antibody or monoclonal antibody is an anti-CTLA4, anti-PD-1, anti-PD-L1, or anti-PD-L2 antibody. In certain implementations, the antibody is a monoclonal anti-PD-1 antibody. In some implementations, the antibody is a monoclonal anti-PD-L1 antibody. In certain implementations, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain implementations, the anti-PD-1 antibody is one or more of pembrolizumab (Keytruda®) or nivolumab (Opdivo®). In certain implementations, the anti-CTLA4 antibody is ipilimumab (Yervoy®). In certain implementations, the anti-PD-L1 antibody is one or more of atezolizumab (Tecentriq®), avelumab (Bavencio®), or durvalumab (Imfinzi®), anti-VEGF (Bevacizumab®).

In certain implementations, the immunotherapy or immunotherapeutic agent is an antagonist (e.g., antibody) against CD80, CD86, LAG3, KIR, TIM3, GAL9, or A2aR. In other implementations, the antagonist is a soluble version of the inhibitory immune checkpoint molecule, such as a soluble fusion protein comprising the extracellular domain of the inhibitory immune checkpoint molecule and an Fc domain of an antibody. In certain implementations, the soluble fusion protein comprises the extracellular domain of CTLA4, PD-1, PD-L1, or PD-L2. In some implementations, the soluble fusion protein comprises the extracellular domain of CD80, CD86, LAG3, KIR, TIM3, GAL9, or A2aR. In one implementation, the soluble fusion protein comprises the extracellular domain of PD-L2 or LAG3.

In certain implementations, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain implementations, the immune checkpoint molecule is a co-stimulatory molecule selected from CD28, inducible T cell co-stimulator (ICOS), CD137,OX40, or CD27. In other implementations, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain implementations, the immunotherapy or immunotherapeutic agent is an agonist of a co-stimulatory checkpoint molecule. In certain implementations, the agonist of the co-stimulatory checkpoint molecule is an agonist antibody and preferably is a monoclonal antibody. In certain implementations, the agonist antibody or monoclonal antibody is an anti-CD28 antibody. In other implementations, the agonist antibody or monoclonal antibody is an anti-ICOS, anti-CD137, anti-OX40, or anti-CD27 antibody. In other implementations, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain implementations, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Components include a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an ASIC. A hardware component may also include programmable logic or circuitry that is temporarily configured by software to perform certain operations.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In implementations in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

### EXAMPLES

### Example 1

Our method is based on mutational signatures. Mutational signatures are patterns of mutations that occur in a genome and can provide insights into the underlying biological processes that lead to those mutations. As was confirmed in tissue, the presence of some mutational signatures, such as Signature 3 (SBS3) can be indicative of HRD.

Since the mutational signatures are derived based on single nucleotide variations (SNVs), the sensitivity of the suggested method is higher compared to the CNV-based methods. Detecting CNVs in cfDNA is more challenging compared to SNVs due to the relatively lower abundance and fragmented nature of cfDNA.

To make the method more robust and increase the sensitivity and specificity one uses a set of training samples to find de novo mutational signatures for a cohort of HRD+ and HRD− samples. The Inventors compute the mutational signature for every test sample and find its decomposition using the denovo HRD+/− signatures. The weights of the decomposition are used as a feature in the ML classifier to compute the probability of a sample being HRD+ or HRD−.

### Example 2

The algorithm requires the training step which consists of several stages:
1. Computing two sets of de novo mutational signatures: each set is derived from a cohort of either HRD+ or HRD− training samples. The mutational signatures are computed from somatic SNVs using these steps:
Context extraction: find one nucleotide upstream and one nucleotide downstream of the mutated position.
Count matrix creation: each row represents a training sample, and each column represents specific mutation type (e.g. A>G, T>C) in a distinct context.
Signature decomposition: use non-negative matrix factorization to discover de novo mutational signatures.
2. For every training sample the Inventors calculated a feature vector composed of the non-negative weights found using non-negative least squares (NNLS). The NNLS minimizes the sum of squared differences between the observed mutational patterns in the sample and the linear combination of de novo mutational signatures.
4. Use the constructed features and the labels of the training samples to train the classification algorithm (e.g. Random Forest classifier).

At the prediction step one computes the feature vector for the test sample as described previously, and then use the pretrained classifier to generate the probability that the test sample is HRD+.

### Example 3

As described in Polak et al. Nature Genetics (2016), SBS3 cancer signature has been proposed as a predictor of defective homologous recombination-based repair. Signature 3 is strongly associated with germline and somatic biallelic inactivation of BRCA1 and BRCA2. In pancreatic cancer, responders to platinum therapy usually exhibit SBS3 mutations. In breast cancer Signature 3 outperforms LOH and LST scores in identifying samples with events in HR-pathway genes: BRCA1, BRCA2, RAD51C, and PALB2 (AUC: 0.8-0.9).

It is appreciated that mutational signatures are obtained from WGS data, which may also involve detection of large rearrangements. While this may be effective in samples such as tissue, the distribution of the mutations and therefore the shape of the signature can be substantially different for a targeted panel in other sample types. The number of somatic SNVs that one can detect in cell free nucleic acids and in their associated target regions is small and insufficient to detect the signature in a single sample. Thus, it is readily appreciated that approaches for WGS are entirely unsuitable for cfDNA, given size and difference.

### Example 4

In order to address the aforementioned limitations in cell free nucleic acids, the Inventors established a platform capable of detection, using de novo signature extraction and a machine learning approach.

For example, one can select samples with a plurality of somatic SNVs (e.g., 10SNVs). Thereafter, a mutational count matrix (96 SBS types×samples) is generated and one extract de novo mutational signatures for HRD+ and HRD− samples. This includes Search for an optimal solution between 1 and 10 mutational signatures. For each rank one can perform 100 independent NMFs of the normalized Poisson resampled input matrices and thereafter identify the optimal factorization rank by simultaneously maximizing the stability and minimizing the reconstruction error.

To reveal the aetiology of the de novo signatures, one can then decompose to known COSMIC signatures including use of a non-negative least squares algorithm.

### Example 5

It should be readily appreciated that computing de novo mutational signatures is widely extendible to different disease context, not limited to cancer. In other instances, each set of de novo signatures is derived from a cohort of either diseased or healthy training samples. The mutational signatures are computed from somatic SNVs, optionally using context etraction such as one nucleotide upstream and one nucleotide downstream of the mutated position, or methylation status, creaetion of a matrix with rows and columns representing training sample, and specific mutation type (e.g. A>G, T>C) in a distinct context. Decomposition is applied including using for example, non-negative matrix factorization, two-layer directed graphical model with one layer of observed random variables and one layer of hidden random variables, or nonnegative quadratic programming to discover de novo mutational signatures.

For each training sample, one can calculate a feature vector composed of weights, including non-negative weights found using non-negative least squares (NNLS), constrained least squares, gradient descent, and coordinate-wise optimization. For NNLS, this is found through minimization of the sum of squared differences between the observed mutational patterns in the sample and the linear combination of de novo mutational signatures.

Thereafter, the constructed features and the labels of the training samples train the classification algorithm (e.g. Random Forest classifier, ensemble learning method for classification, regression, linear models as base estimators in random forests, in particular multinomial logistic regression and naive Bayes classifiers).

At the prediction step one computes the feature vector for the test sample as described previously, and then use the pretrained classifier to generate the probability that the test sample is disease or not diseased.

### Example 6

As described, the error-prone nature of NHEJ leads to characteristic “genomic scars” (sometimes termed BRCAness). Here, one can measure the genomic scars that occur in HRD cells and use these measures as biomarkers for predicting response to targeted PARPi therapy. But unlike other techniques, the de novo signatures generated by the methods herein are capable of use with cfDNA samples, which otherwise would be too scarce for detection and informative analysis without techniques such as WGS. While these techniques largely improve upon conventional techniques, other measurements (e.g., LOH, LST, TAI) may nevertheless provide further information for analysis and use in an ensemble combining the SBS signatures described herein and other detection modalities.

### Example 7

For a given signature, the systems and methods described can subsequently be utilized in quantitative analysis through comparison of the methylation status between the wild-type and mutant alleles i.e., measuring the degree of methylation at the specific CpG sites (including CpG sites identified using single site methylation (SSM)) within the signature for both alleles Thereafter, one can then expressing this comparison as a ratio. In some application one can then use this as an additional input parameter for an ensemble model.

1. A method, comprising:
determining context of at least one mutated position from of a plurality of nucleic acids each obtained from a plurality of samples;
creating at least one matrix comprising a sample and the at least mutation context;
processing the at least one matrix to generate one or more mutational signatures;
determining at least one metric for each of the plurality of samples.

2. The method of claim 1, wherein the at least one metric trains a classification algorithm.

3. The method of claim 2, wherein the training comprises a linear classifier, neutral network, decision tree, kernel estimation, support vector machine.

4. The method of claim 2, wherein the trained classification algorithm calculates a probability of a test sample being HRD positive or HRD negative.

5. The method of claim 1, wherein processing the at least one matrix comprises non-negative matrix factorization.

6. The method of claim 1, the at least one metric comprises a feature vector comprising non-negative weights (NNW) determined using non-negative lease squares (NNLS).

7. The method of claim 1, wherein determining the context of at least one mutated position comprises identifying at least one nucleotide upstream and one nucleotide downstream of the mutated position.

8. The method of claim 1, wherein creating at least one matrix comprises one or more rows and one or more columns.

9. The method of claim 1, wherein creating at least one matrix comprises a row comprising one or more training samples and columns comprising a single base mutation in the determined context.

10. The method of claim 1, comprising obtaining a sample from a human subject.

11. The method of claim 9, wherein the sample comprises cell free DNA (cfDNA).

12. The method of claim 1, comprising selecting a treatment based on the determination of at least one metric.

13. The method of claim 11, wherein the treatment is a poly adenosine diphosphate (ADP) ribose polymerase (PARP) inhibitor.

14. The method of claim 11, comprising administration of the treatment to a human subject.

15. A method, comprising:
determining context of at least one mutated position from of a plurality of nucleic acids each obtained from a plurality of samples;
creating at least one matrix comprising a sample and the at least mutation context;
processing the at least one matrix to generate one or more mutational signatures;
determining at least one metric for each of the plurality of samples;
training a classification algorithm with the at least one metric;
calculating a probability of a test sample being HRD positive or HRD negative using the trained classification algorithm.

16. A method, comprising:
determining context of at least one mutated position from of a plurality of nucleic acids each obtained from a plurality of HRD positive or HRD negative samples, wherein the context comprises one nucleotide upstream and one nucleotide downstream;
creating at least one matrix comprising a sample and the at least mutation context;
processing the at least one matrix to generate one or more mutational signatures using non-negative matrix factorization;
determining at least one metric for each of the plurality of samples, wherein the at least one metric comprises a feature vector comprising non-negative weights (NNW) determined using non-negative lease squares (NNLS);
training a classification algorithm with the at least one metric;
calculating a probability of a test sample being HRD positive or HRD negative using the trained classification algorithm.

17. A method, comprising:
determining, by computing system and implementing a predictive model, individual probabilities of a homologous recombination repair deficiency being present in individual samples in a plurality of samples; and
determining, by the computing system and based on the individual probabilities, a probability to indicate a homologous recombination repair deficiency being present with respect to a given subject.

18. The method of claim 1, comprising:
determining, by the computing system, a responsiveness to treatment with respect to a group of subjects, wherein cancer is detected in the group of subjects and the treatment is provided to treat the cancer; and
determining, by the computing system, the plurality of samples that correspond to subjects having a homologous recombination repair deficiency based on the responsiveness of a portion of the group of subjects to the treatment.

19. The method of claim 17, wherein the treatment is a poly adenosine diphosphate (ADP) ribose polymerase (PARP) inhibitor.
